(19) United States
(12) Patent Application Publication
(10) Pub. No.: US 2007/0225602 A1
(43) Pub. Date: Sep. 27, 2007

(54) Methods

(75) Inventors: Michelle Heaton, Leicestershire (GB); Richard Fulton Butterwick, Leicestershire (GB)

Correspondence Address: FULBRIGHT & JAWORSKI, LLP, 1301 MCKINNEY, SUITE 5100, HOUSTON, TX, 77010-3095, US

(73) Assignee: Mars, Inc., McLean, VA (US)

(21) Appl. No.: 10/582,026

(22) PCT Filed: Dec. 10, 2004
(86) PCT No.: PCT/GB04/05201

§ 371 Date: Mar. 6, 2007

(30) Foreign Application Priority Data
Dec. 11, 2003 (GB) 0328747.1

(51) Int. Cl. A61B 6/14

(52) U.S. Cl. 600/436

(57) ABSTRACT

The present invention relates to a method of diagnosing feline odontoclastic resorptive lesions involving taking no more than seven radiographic views of teeth from a feline animal. At least one view is of each of the mandibular third premolar teeth.

Feline odontoclastic resorptive lesions (FORL) are one of the most common oral conditions in domestic cats, with prevalence increasing with age. During disease, odontoclasts mediate root resorption that is combined with the deposition of bone or cementum-like tissue by blast cells in an attempt to repair the damage. Root tissue is progressively lost, undermining the crown that may eventually break.

The prevalence of FORL in the domestic cat population has been reported to be 2-75%. The variation can be accounted for by differences in age, source of the population studied and diagnostic methods used.

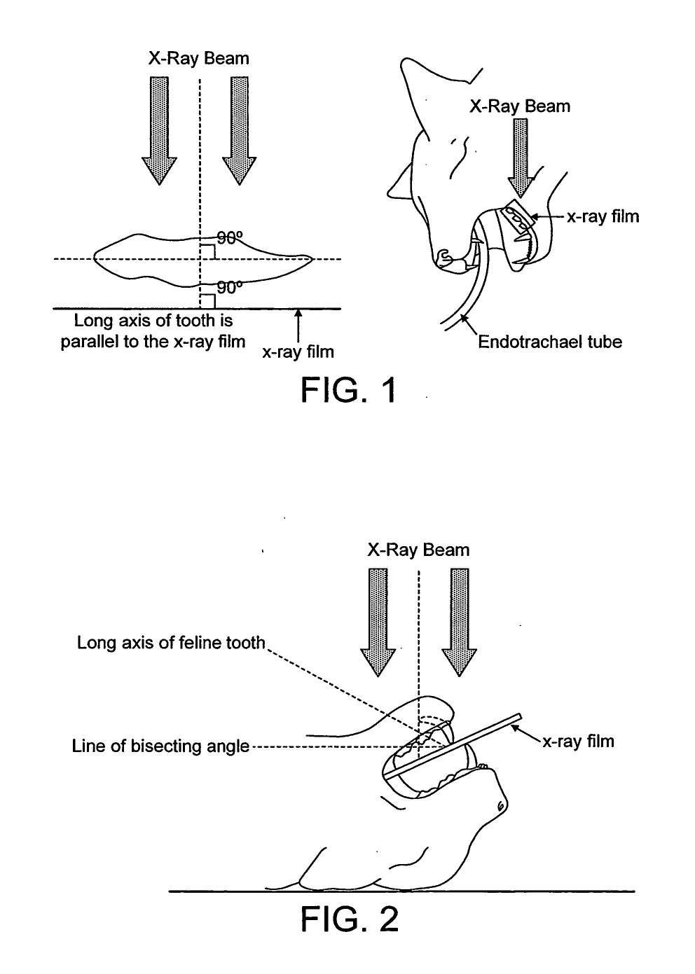

Diagnosis of FORL is made by clinical examination (visual inspection and tactile exploration) or by a combination of clinical and radiographical examination. Clinical examination alone can only detect FORL above the gingival margin, missing lesions occurring in the root of the tooth and therefore radiographic studies are recommended. Radiographic changes are usually discrete and therefore clarity and detail are essential. For a dental radiograph to be diagnostic, it should be an accurate representation of the size and shape of the tooth without superimposition of adjacent structures. Therefore intraoral radiographic techniques are required; a parallel technique (FIG. 1) for the mandibular premolars and molars, and a bisecting angle technique (FIG. 2) for all other teeth. Whilst the parallel technique requires minimal radiographic training, the bisecting angle method is technically difficult and requires extensive training and experience, placing it beyond the reach of many veterinary practices.

‘Full mouth radiographs’ is the term used to describe a series of dental films where each tooth of the dentition is accurately depicted in at least one view.

This is the gold-standard for FORL diagnosis, but requires 8-10 separate films that may take 15-30 minutes to obtain, during which time the subject must be fully anaesthetised. The eight basic views (each view indicated in square bracket, below) are as follows:

Incisor view in the upper jaw (maxillary incisor teeth) [1]

Lateral view for each of the canines of the upper jaw (right maxillary canine and left maxillary canine) [2]

Left and right maxillary premolar and molar views (right maxillary premolar and molar, and left maxillary premolar and molar) [2]

Mandibular incisor and canine view (mandibular incisors and canines) [1]

Left and right mandibular premolar and molar views. (right mandibular premolar and molar, and left mandibular premolar and molar) [2]

In addition, lateral mandibular canine views (2 films) may be required.

It is disputed which teeth are most commonly affected with FORL. Ingham and others (2001), in Prevalence of odontoclastic resorptive lesions in a population of clinically healthy cats, Journal of Small Animal Practice 42, 439-443 have reported in their study that the teeth most commonly affected with FORL were the third mandibular premolars. However, in a later study, Van Wessum, R., Harvey, C. E. & Hennet, P. (1992), Feline dental resorptive lesions, Prevalence patterns, Veterinary Clinics of North America: Small Animal Practice 22, 1405-1416 report that the mandibular and maxillary fourth premolars are the most commonly affected with FORL. It was therefore of interest to determine if the FORL status of two teeth alone provided a reliable indicator of overall FORL status. The present study aimed to determine if FORL could be accurately diagnosed from the left and right mandibular premolar and molar views (2 intraoral films and parallel radiographic technique) alone.

Accordingly, the present invention provides a method of diagnosing FORL, comprising taking no more than seven radiographic views of teeth from a feline animal, wherein at least one view is of each of the mandibular premolar teeth.

The invention comprises taking two, three, four, five, six or seven radiographic views of teeth from the mouth of a feline animal, wherein at least one view is of each of the manidubular third premolar teeth.

Preferably, at least two radiographic views are parallel views. Most preferably, these two parallel views are of the mandibular third premolar teeth.

The parallel technique is used usually for the mandibular premolars and molars. Details with regard to use the parallel technique is described herein with reference to FIG. 1. For the other teeth, a bisecting angle technique is used. A discussion with regard to use of the bisecting angle technique is disclosed herein with reference to FIG. 2.

It should be noted that oral examination, charting and radiology in accordance with the invention are usually (and may exclusively be) performed by a veterinary nurse and/or a dental technician whom are not veterinary specialists in dentistry. Accordingly, the radiographic results do not make it immediately possible to decide on a particular course of medical treatment.

The present invention particularly relates to a method of obtaining radiographic data of feline teeth, the method comprising taking no more than seven radiographic views of teeth from a mouth of a feline animal, wherein at least one view is of each of the mandibular third premolar teeth. This method may comprise taking two, three, four, five, six or seven radiographic views of teeth from the mouth of a feline animal.

Preferably, at least two radiographic views are parallel views. Most preferably, these two parallel views are of the mandibular third premolar teeth.

The present invention also relates to the use of no more than seven radiographic views of teeth from the mouth of a feline animal, wherein at least one view is of each of the mandibular third premolar teeth, and the manufacturer for a tool for diagnosing FORL.

This use may comprise two, three, four, five or six radiographic views.

Preferably, at least two radiographic views are parallel. Most preferably, the two parallel views are of the mandibular premolar teeth.

The present invention also relates to dental treatment performed as the result of the diagnosis of FORL in the teeth of the feline animal, following a method according to the present invention. Accordingly, the method of the invention relates to a method as described herein according to the invention followed by the appropriate medical treatment.

The present invention relates to the number of radiographic views needed in order to determine an initial result for the presence or absence of FORL. If the initial less than seven radiographic views provide the result that FORL is present, it may be necessary or appropriate to take further radiographic views of additional teeth. Thus, after an initial result is obtained, further radiographic views, to a total of eight or over may be taken. However, the advantage of the present invention is that less than eight views are initially required in order to determine whether further time, money and effort are required by taking additional views.

The present invention is described with reference to the figures, in which:

FIG. 1 shows the Parallel Technique

The parallel technique is used for the mandibular premolars and the molars. The patient is placed in lateral recumbency (with the side to be radiographed uppermost). The film is placed between the tongue and the teeth and pushed as far down into the sublingual fossa as possible. The X-ray beam is then directed from lateral to medial at right angles to the long axis of the tooth. The resulting image of the tooth has very little magnification or distortion. Due to the anatomy of the oral cavity, this technique is only possible in the mandibular premolar and molar regions.

FIG. 2 shows the bisecting angle technique

The Bisecting angle technique is required to minimise distortion when taking radiographs of the teeth in the upper jaw and at the mandibular incisors and canines. The film is positioned at an angle behind the tooth in question. To avoid foreshortening or elongation of the image, an imaginary plane is drawn half way between the plane of the film and a plane through the long axis of the tooth, i.e. at the bisecting angle, and the X-ray beam is directed perpendicular to this plane. In this way, both sides of the triangles formed are the same length and the resulting image of the tooth is similar to the real tooth.

FIG. 3 shows the side view of a cat skull with teeth shown.

FIG. 4 shows the numbering of the maxillary and mandibular teeth

The invention is now described with and referenced to the following example:

### EXAMPLE

Introduction

The FORL status (presence or absence of odontoclastic resorptive lesions) of 423 clinically healthy cats was determined based on radiographic findings in a series of full mouth radiographs (8 views). This status was compared to FORL status based on evaluation of only two views, namely the right and left mandibular premolar and molar views. Using the FORL status of the right and left third mandibular premolars (307 and 407) alone correctly predicted overall FORL status in 93.4% cats. Overall FORL status can therefore be confidently diagnosed in 9/10 cats by assessing FORL status in just two teeth (307, 407) using two films, which has benefits for the cat (less anaesthetic time and reduced exposure to radiation) and owner (reduced cost of screening).

Materials and Methods

Subjects

Four hundred and twenty-three cats (mean age 4.44±SEM 0.14 years, age range 1-14 years; 414 domestic shorthaired and 9 British Blue) were studied, consisting of 243 females (45 entire, 198 neutered) and 180 males (9 entire, 171 neutered). The cats were all bred and maintained by the WALTHAM Centre for Pet Nutrition, housed in groups of 20-25 in purpose-built, environmentally enriched surroundings. All cats had been fed a variety of dry and wet commercially available pet foods throughout their lives, and were clinically healthy as determined by a full clinical physical examination. This study complies with Waltham's rigorous ethical policy.

Study Design

FORL status was assessed on a single occasion. Each cat underwent a thorough oral examination and a full mouth series of dental radiographs were taken under general anaesthesia at the time of a scheduled dental cleaning. The oral examination, charting and radiography were performed by a listed veterinary nurse and a dental technician who had received training from, and worked under the supervision of, a European veterinary specialist in dentistry. Radiographic interpretation was performed by the specialist. Where indicated, additional dental treatment was also performed.

General Anaesthesia

The cats were pre-medicated with a combination of 0.4 mg/kg butorphanol (Torbugesic; Fort Dodge Animal Health) and 0.05 mg/kg medetomidine (Domitor; Pfizer) by subcutaneous injection, and anaesthesia was induced by masking with 2 per cent isoflurane (IsoFlo Vet; Schering-Plough Animal Health) in oxygen. A cuffed endotracheal tube was placed and anaesthesia was maintained using isoflurane in oxygen and a non-breathing Ayre's T-piece anaesthetic circuit. The medetomidine sedation was reversed once treatment had been completed by the intramuscular injection of 0.125 mg/kg atipamezole (Antisedan; Pfizer).

FORL Status

Each cat underwent a full clinical oral examination, including gingivitis scoring, and a full mouth series of dental radiographs were obtained. The oral examination consisted of visual inspection and tactile exploration of the tooth surfaces using a dental explorer. In addition, the periodontium was investigated using a blunt-ended periodontal probe.

Radiography consisted of eight views, using intra-oral placement of periapical size dental film and a dental x-ray machine. Mandibular premolar and molar views were obtained using a parallel technique, whilst all other views were taken using a bisecting angle technique.

The presence of FORL was determined by radiographic features such as loss of integrity of periodontal ligament space, irregularities in the root surface, radiolucent areas within the root dentine often extending into the crown dentine, and replacement of root substance by bone-like tissue. Teeth were classified as missing if they were absent on the radiograph. Some teeth were missing clinically, but the resorbing roots were present on the radiograph; these teeth were classified as teeth affected by FORL rather than missing. Teeth were also identified (306, 406) that are not recognised as part of the normal feline dentition and were disregarded from the data set.

Model and Statistics

Using the eight-film, full screening procedure, the true FORL status of each cat was determined. In addition, the test FORL status was determined using the results from 307, 407 on the two premolar and molar views alone.

Missing teeth could not be assumed to be missing due to FORL, and the following rationale was applied. If 307 and 407 were both present they were scored as usual. If 307 and 407 were both missing completely (and the resorbing roots were not radiographically visible), it could not be assumed that the loss was due to FORL and so this situation was scored as FORL absent. Where either 307 or 407 were missing, but there was no evidence of FORL in the other one, again this was scored FORL absent.

The true FORL status (8 film) was then compared to the test FORL status (2 film) from simple cross-tabulation tables.

Results

The prevalence rate of FORL in the sample of cats was 30.7% (table 1). The true FORL status was correctly predicted using two films alone in 93.4% cats (table 1).

Table 1: Comparison of FORL status in 423 cats using two methods. True FORL status was determined using a full mouth (8 film) series, whilst test FORL status was determined using a reduced (2 film) series of dental radiographs.

TABLE 1

| | |
|---|---|
| Number of cats | True FORL status (8 film) |

| | | |
|---|---|---|
| (n = 423) | FORL+ | FORL− |

| | | | |
|---|---|---|---|
| Test FORL status | FORL+ | 102 | 0 |
| (2 film) | FORL− | 28 | 293 |

Discussion

The current study has shown that FORL status can be diagnosed in 9/10 cats using just two radiographs. This finding has important implications for both the cat, which is exposed to shorter anaesthesia and less radiation, and the veterinary practice, since the investigation can be conducted rapidly and at a reduced cost to the client. The technique is therefore suitable as a rapid screening procedure for FORL that can be adopted by all practices. These two views require intraoral positioning of dental film, but can be taken with a conventional veterinary radiographic unit.

The adoption of a simplified, standardised technique for FORL diagnosis would assist in the estimation of prevalence of the disease, which has been reported to be anywhere between 2-75%.

Conclusion

This study provides a rapid screening technique for FORL that will confidently diagnose 9/10 cats and that can be adopted by all veterinary practices. The radiographic technique is easy to learn and does not require purchase of specialised equipment as these views can be taken with a routine veterinary X-ray unit. It is recommended that left and right mandibular premolar and molar views are taken of all cats anaesthetised for dental treatment.

1. A method of diagnosing FORL comprising taking no more than seven radiographic views of teeth from the mouth of a feline animal, wherein at least one view is of each of the mandibular third premolar teeth.

2. A method, as claimed in claim 1, comprising taking no more then four radiographic views of teeth from the mouth of a feline animal.

3. A method, as claimed in claim 1, comprising taking no more than two radiographic views of teeth from the mouth of a feline animal.

4. A method, as claimed in claim 1 wherein at least two radiographic views are parallel views.

5. (canceled)

6. (canceled)
